(19) United States
(12) Patent Application Publication
(10) Pub. No.: US 2018/0140787 A1
(43) Pub. Date: May 24, 2018

(54) PROPELLANT BASED METERED DOSE INHALER AND FOOD APPLICATORS AND APPLICATORS

(71) Applicant: Island Breeze Systems CA, LLC, Oakland, CA (US)

(72) Inventor: Michael S. Hartman, Millbrae, CA (US)

(21) Appl. No.: 15/575,058

(22) PCT Filed: May 16, 2016
(86) PCT No.: PCT/US16/32777

§ 371 Date: Nov. 17, 2017

Related U.S. Application Data
(60) Provisional application No. 62/165,098, filed on May 21, 2015.
(60) Provisional application No. 62/208,475, filed on Aug. 21, 2015.

(51) Int. Cl. A61M 15/00

(52) U.S. Cl. CPC A61M 15/009; A61M 15/0025; A61M 16/0866; A61M 15/0081; A61M 15/0066; A61M 15/008

(57) ABSTRACT

Provided herein are embodiments related to metered dose inhalers and/or food applicators and/or applicators.

### RELATED APPLICATIONS

This application claims priority to U.S. Provisional Applications Ser. No. 62/165098, filed May 21, 2015, and Ser. No. 62/208,475, filed Aug. 21, 2015, each of which is hereby incorporated by reference in their entireties.

### BACKGROUND

Provided herein are various metered dose inhalers, associated devices, applicators, and food applicators.

### Field

The present application relates to metered dose inhaler related products, food applicators, and applicators.

### SUMMARY

In some embodiments, a metered dose inhaler is provided. The MDI comprises an actuator that comprises a receiver for a canister; an orifice; and a channel fluidly connecting the receiver for the canister to the orifice such that a fluid emitted from the canister is channeled through the channel and out of the orifice to and out of a mouth piece, wherein the mouth piece comprises an opening. The opening is at least one of: a) between about 1 mm to less than 1.5 cm in diameter, b) between about 1 cm² and 7.85*10⁻³ cm² in area, or c) located a distance of more than about 5 mm and less than about 10 cm from the nozzle. In some embodiments, a method of restricting a dose supplied by a metered dose inhaler is provided. The method comprises passing a dose supplied by a metered dose inhaler through a restricted opening, wherein the restricted opening has a diameter of less than 1.5 cm, and wherein a level of discomfort due to the dose is thereby decreased, relative to a level of discomfort that occurs for administration of the dose through an opening that has a diameter of more than 1.5 cm.

In some embodiments, a method of restricting a dose supplied by a metered dose inhaler is provided. The method comprises passing a dose supplied by a metered dose inhaler through a restricted opening, wherein the restricted opening has an area of less than 1.5 cm², and wherein a level of discomfort due to the dose is thereby decreased relative to a level of discomfort that occurs for administration of the dose through an opening that has an area of more than 1.5 cm².

In some embodiments, a method for decreasing discomfort from an application of a dose from a metered dose inhaler is provided. The method comprises providing an altered opening over a mouth piece for a metered dose inhaler. The altered opening is at least one of: a) between about 1 mm to less than 1.5 cm in diameter, b) between about 1 cm² and 7.85*10⁻³ cm² in area, or c) located at distance of more than about 5 mm and less than about 10 cm from a nozzle within the metered dose inhaler.

In some embodiments, an applicator is provided. The applicator comprises a canister retaining wall configured to receive and retain a canister, wherein the canister is a metered dose inhalation canister; and a base comprising a hole. The hole is configured a) to receive a stem of the metered dose inhalation canister in a manner such a force can be applied between the base and a back of the canister, which will result in an actuation of the metered dose inhalation canister, and b) to allow for a dose from the metered dose inhalation canister to pass through the hole. The base is operably coupled to the canister retaining wall such that a force applied to the canister retaining wall is transferred to the base.

In some embodiments, a method of applying a hemp and/or cannabis extract onto a food product is provided. The method comprises providing a metered dose inhalation canister containing a hemp and/or cannabis extract; and actuating the canister so that a dose that is released from the canister upon actuation is distributed to a food product.

In some embodiments, a cap for a metered dose inhaler is provided. The cap is configured to fit over an opening of a mouth piece for a metered dose inhaler, the cap comprising a hole, wherein the hole is at least one of: between about 1 mm to less than 1.5 cm in diameter; between about 1 cm² and 7.85*10⁻³ cm² in area; or located a distance of more than about 5 mm and less than about 10 cm from a nozzle in the metered dose inhaler when the cap is attached to the opening of a mouth piece of the metered dose inhaler.

In some embodiments, an insert for a metered dose inhaler is provided. The insert is configured to fit within an opening of a mouth piece for a metered dose inhaler so as to effectively seal the opening of the mouth piece against an outer perimeter of the insert. The insert comprises a hole, wherein the hole is at least one of: between about 1 mm to less than 1.5 cm in diameter, between about 1 cm² and 7.85*10⁻³ cm² in area, or located a distance of more than about 5 mm and less than about 10 cm from a nozzle in the metered dose inhaler when the insert is inserted in place in the opening of the mouth piece of the metered dose inhaler.

In some embodiments, a kit is provided. The kit comprises at least one of: a) a cap configured to fit over an opening of a mouth piece for a metered dose inhaler, the cap comprising a hole, wherein the hole is at least one of: between about 1 mm to less than 1.5 cm in diameter, between about 1 cm² and 7.85*10⁻³ cm² in area, or located a distance of more than about 5 mm and less than about 10 cm from a nozzle in the metered dose inhaler when the cap is attached to the opening of a mouth piece of the metered dose inhaler; or b) an insert configured to fit within an opening of a mouth piece for a metered dose inhaler so as to effectively seal the opening of the mouth piece against an outer perimeter of the insert, the insert comprising a hole, wherein the hole is at least one of: between about 1 mm to less than 1.5 cm in diameter, between about 1 cm² and 7.85*10⁻³ cm² in area, or located a distance of more than about 5 mm and less than about 10 cm from a nozzle in the metered dose inhaler when the insert is inserted in place in the opening of the mouth piece of the metered dose inhaler; and c) at least one of: a canister that contains a cannabis and/or hemp extract; or a metered dose inhaler.

### BRIEF DESCRIPTION OF THE DRAWINGS

FIG. 1 depicts a cross-sectional diagram of a metered dose inhaler.

FIG. 2 depicts a diagram of some embodiments of a metered dose inhaler with a restricted hole or opening of some of the embodiments provided herein.

FIG. 3 depicts a diagram of some embodiments of a metered dose inhaler indicating a distance l from the orifice to the hole that is the mouth piece.

FIG. 4 depicts a diagram of some embodiments of a food applicator (a device for propellant based application of a formulation onto various substances, such as food).

FIG. 5 depicts a diagram of some embodiments of a cap for a metered dose inhaler with a restricted hole.

FIGS. 6A and 6B depict an exploded and assembled arrangement for some embodiments of a food applicator.

### DETAILED DESCRIPTION OF VARIOUS EMBODIMENTS

Provided herein are devices that provide or result in a more comfortable subject experience when a metered dose inhaler (“MDI”) is used to administer a formulation.

Traditional HFA pMDI product development is focused on highly pure, reproducible and efficient aerosols. Characteristics typically embody a Fine Particle Fraction, Mass Median Aerodynamic Diameter, delivered dose content uniformity through product life and loss of product potency on stability. Due to the narrow therapeutic index of the vast majority of pharmaceutical actives (steroids, beta-blockers, anticholinergics, etc.) product development is often not done in vivo and comfort typically takes a lower level of importance over the need to deliver a therapeutic dose. Often, any harshness to the respiratory tract from an MDI device is ignored and listed as a side-effect of the product with the aforementioned product characteristics taking priority.

Some of the embodiments provided herein relating to MDIs (and related aspects) allow for a much smoother aerosol experience by screening out large propellant droplets to produce an aerosol that, in some embodiments is smoother and results in less coughing. In other words, some embodiments provided herein are modifications of MDIs to provide for a more comfortable user experience with a MDI. In some embodiments, this can achieved by varying the opening for the MDI and/or the distance from the nozzle of the actuator to the opening of the MDI.

Some embodiments provided herein are related to food applicator devices and/or methods. In some embodiments, these devices allow for a user to employ a propellant based substance (such as is often used in canisters for metered dose inhalers) to various substances, such as food.

These two concepts are discussed in more detail below.

### Metered Dose Inhaler Embodiments

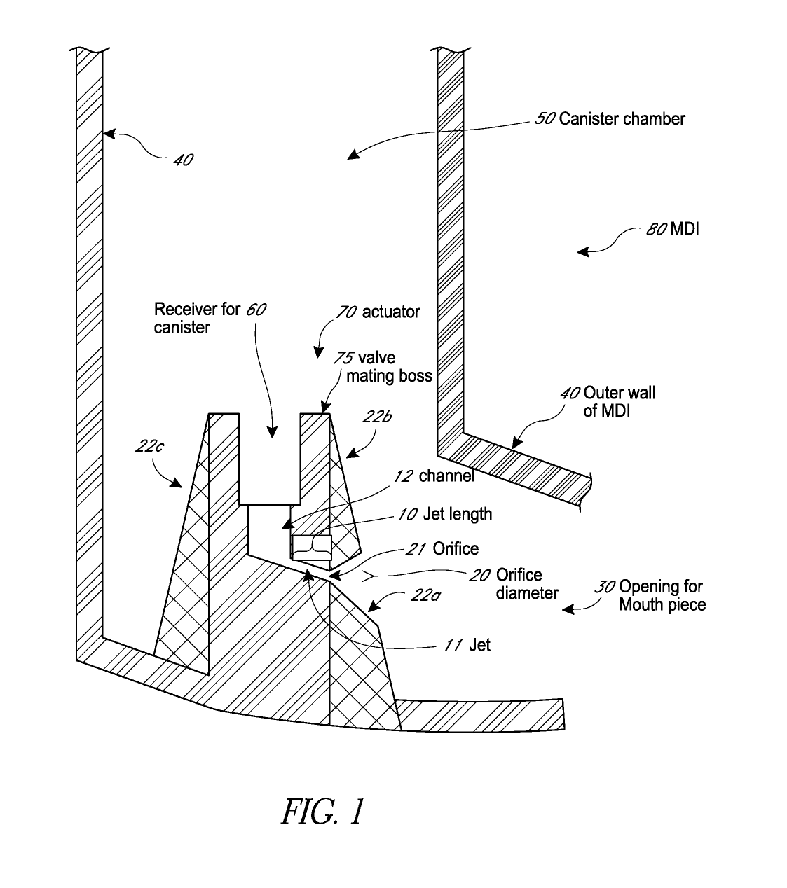

FIG. 1 depicts some embodiments of a metered dose inhaler 80 for the delivery of a metered dose inhaler formulation. A MDI can include a chamber 50, configured for receiving the canister that contains the formulation, an actuator 70, which can include a valve mating boss 75 for the canister, a channel 12 which allows the formulation from the canister to pass through it, through the jet 11, out an orifice 21 of the actuator (or actuator orifice), through the opening for the mouth piece 30, to pass out of the mouth piece itself (not shown). Often, especially in traditional MDIs, the outer wall 40 of the MDI can form the mouthpiece and the chamber 50. In some situations, altering the jet length 10 and/or the actuator orifice diameter 20 can result in a more desirable experience for the user for receiving the formulation. In some embodiments, any of the formulations provided herein can be applied in the device of FIG. 1.

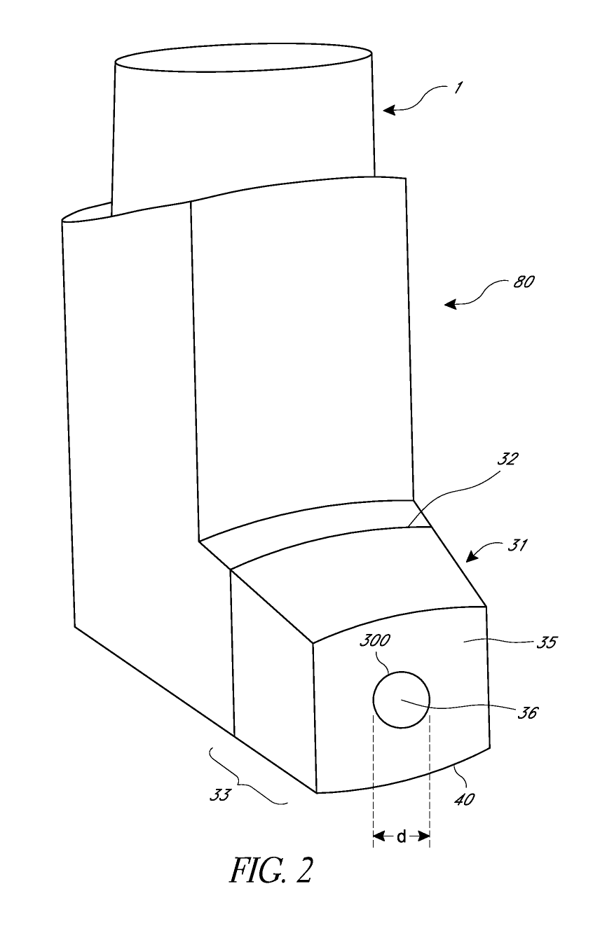

In some embodiments, the typical MDI can be further modified so that it includes a hole of a desired size for its mouth piece 30. As shown in FIG. 2, in some embodiments, the MDI 80 can include a restricted opening 300 as the mouth piece. Contrary to systems in which full dosing is essential, the restricted opening 300 serves to increase the comfort for the user for doses administered by inhalation.

In some embodiments, the restricted opening 300 has a diameter “d” ranging from size of 1 mm to 1.5 cm. In some embodiments, the diameter is between 5 mm and 1.4 cm, 6 mm and 1.3 cm, 8 mm and 1.3 cm, 1 cm and 1.3 cm, 1 cm and 1.3 cm, 1 cm and 1.3 cm, 1 cm and 1.3 cm, for example. In some embodiments, the size is between 1 mm and 10 mm, for example, about 1, 2, 3, 4, 5, 6, 7, 8, 9, or 10 mm in diameter.

In some embodiments, the restricted opening 300 has a first area 36, which is a fraction (less than the same size) of the second area 35 that is typical in an unrestricted MDI. That is, the first area 36 is smaller than the second area 35, and thus, the opening 300 is restricted. In some embodiments, the first area 36 is less than 80% the size of the second area 35, for example, the first area 36 is 80%, 70%, 60%, 50%, 40%, 30%, 20%, 10%, 5%, 1% or less of the size of the second area 35. In some embodiments, the second area 35 can be that of an opening for a mouthpiece in an unrestricted MDI (for example, the opening for the mouth piece 30 in FIG. 1). In some embodiments, the restricted opening has an area that is less than 1.5 cm² for example 1, 0.9, 0.8, 0.7, 0.6, 0.5, 0.4, 0.3, 0.2, or less than 0.1 cm².

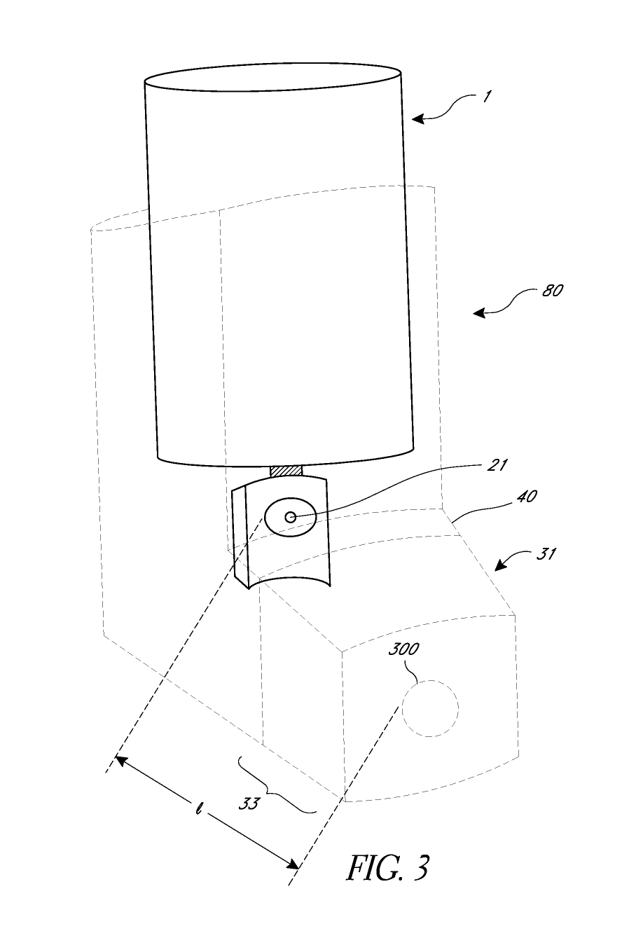

As shown in FIG. 3, in some embodiments, a distance 1 between the orifice 21 and the opening for the mouth piece (which can be, but need not be in all embodiments a restricted opening 300) can be varied for superior comfort to a user. In some embodiments, the distance 1 is from 5 mm to 10 cm. In some embodiments, the distance 1 is 5, 6, 7, 8, 9, 10, 11, 12, 13, 14, 15, 16, 17, 18, 19, or 20 mm. In some embodiments, the distance 1 is 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15 cm long.

In some embodiments, the size of the restricted opening 300 and/or the length 1 can be the result of the configuration of the outer wall of the MDI 40. Thus, in some embodiments, the dimensions 1 and d are due to the shape of the outer wall and are in integral part of the MDI 80 itself.

In some embodiments, the dimensions of d and/or 1 can instead be the result of a cap 31 that can be attached or slid over a standard opening for a mouth piece 30 of an MDI 80. Thus, in some embodiments, a cap 31 can have its length 33 increased and/or decreased, thereby altering the length 1 between the orifice 21 and the opening. In some embodiments, the length of the cap 33 is 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, or 10 cm and/or is sufficient that when placed on an MDI, it results in a length 1 of 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, or 10 cm.

In some embodiments, the cap 31 can be kept in place by a compression or frictional forces between the inside of the cap 31 and an outer surface of the outer wall 40 of the MDI. In some embodiments, the cap can be retained by an adhesive. In some embodiments, a set of ridges and/or grooves can be used to allow the cap to snap into place on the outside of the outer wall of the MDI 40. In some embodiments, the cap 31 can be kept in place via a piece of elastic.

In some embodiments, the cap 31 need not fit around the entire circumference of the opening for the mouth piece, as long as an adequate seal is made to direct an adequate amount of the material from the canister through the restricted opening 300. In some embodiments, the cap 31 comprises an elastic band that can be placed over the opening for the mouth piece 30 and around the opposite side of the MDI 80, so as to keep the band in place over the opening for the mouth piece 30. In some embodiments, the band will have a hole 300 that will be the restricted opening hole, when placed over the opening for the mouth piece 30. In any of the embodiments herein with a hole 300, the hole can be replaced with a mesh that allows an equivalent amount of airflow through it as the hole of a given area and/or diameter provided herein. In some embodiments, a filter or fabric covering over a normal sized mouth piece can also be employed.

In some embodiments, a cap for a metered dose inhaler is provided, the cap being configured to fit over an opening of a mouth piece for a metered dose inhaler, the cap comprising a hole, wherein the hole is between about 1 mm to 3 cm in diameter. In some embodiments of the cap, the hole is between about 7.07 cm² and 7.85*10⁻³ cm² in area. In some embodiments, a cap for a metered dose inhaler is provided, the cap being configured to fit over an opening of a mouth piece for a metered dose inhaler, the cap comprising a hole, wherein the hole is between about 1 mm to less than 1.5 cm in diameter. In some embodiments of the cap, the hole is between about 1.5 cm² and 7.85*10⁻³ cm² in area, for example 1 cm² and 7.85*10⁻³ cm² in area. In some embodiments, a cap for a metered dose inhaler is provided, the cap being configured to fit over an opening of a mouth piece for a metered dose inhaler, the cap comprising a hole, wherein the hole is between about 1 mm to 1.5 cm in diameter, for example 1 mm to 1 cm in diameter. In some embodiments of the cap, the hole is between about 1 cm² and 7.85*10⁻³ cm² in area. In some embodiments of the cap, the hole is located a distance of more than about 5 mm and less than about 10 cm from a nozzle in the metered dose inhaler when the cap is attached to the opening of a mouth piece of the metered dose inhaler.

In some embodiments, a series, set, or kit of caps is provided. The set of caps can comprise increasing or decreasing sizes for the restricted opening 300 so as to lower the dose of the product to the desired level of comfort. Thus, in some embodiments, a set of caps can be supplied together. In some embodiments, the restricted opening 300 can be adjustable. In some embodiments, the cap 31 can have areas that can be pushed through or torn off so as to allow someone using the MDI to select the size of the desired restricted opening 300 by punching out a hole. In some embodiments, by using progressively smaller restricted openings, one can provide progressively lower doses from the same MDI.

In some embodiments, instead of an outer cap 31, an insert can be employed within the opening of the mouthpiece 30. For example, rather than a cap an insert configured to be positioned inside the actuator to produce any one or more of the above noted d and/or l dimensions can be employed. In some embodiments, the insert being configured to fit within an opening of a mouth piece for a metered dose inhaler so as to effectively seal the opening of the mouth piece against an outer perimeter of the insert, the insert comprising a hole, wherein the hole is between about 1 mm to 3 cm in diameter. In some embodiments, the insert being configured to fit within an opening of a mouth piece for a metered dose inhaler so as to effectively seal the opening of the mouth piece against an outer perimeter of the insert, the insert comprising a hole, wherein the hole is between about 1 mm to 1.5 cm in diameter, 1 mm to 1 cm in diameter. In some embodiments of the insert, the hole is between about 7.07 cm² and 7.85*10⁻³ cm² in area, for example 1 cm² and 7.85*10⁻³ cm². In some embodiments of the insert, the hole is located a distance of more than about 5 mm and less than about 10 cm from a nozzle in the metered dose inhaler when the insert is inserted in place in the opening of the mouth piece of the metered dose inhaler. In some embodiments of the insert, the hole is between about 1 cm² and 7.85*10⁻³ cm² in area.

In some embodiments, a kit is provided, the kit comprising a cap configured to fit over an opening of a mouth piece for a metered dose inhaler, the cap comprising a hole, wherein the hole is between about 1 mm to 1.5 cm in diameter, for example 1 mm to 1 cm in diameter. In some embodiments of the kit comprising the cap, the hole is between about 1 cm² and 7.85*10⁻³ cm² in area. In some embodiments of the kit comprising the cap, the hole is located a distance of more than about 5 mm and less than about 10 cm from a nozzle in the metered dose inhaler when the cap is attached to the opening of a mouth piece of the metered dose inhaler.

In some embodiments, a kit is provided, the kit comprising an insert configured to fit within an opening of a mouth piece for a metered dose inhaler so as to effectively seal the opening of the mouth piece against an outer perimeter of the insert, the insert comprising a hole, wherein the hole is between about 1 mm to 1.5 cm in diameter, for example 1 mm to 1 cm in diameter. In some embodiments, a kit is provided, the kit comprising an insert configured to fit within an opening of a mouth piece for a metered dose inhaler so as to effectively seal the opening of the mouth piece against an outer perimeter of the insert, the insert comprising a hole, wherein the hole is between about 1 mm to 1.5 cm in diameter, for example 1 mm to 1 cm in diameter. In some embodiments of the kit with the insert, the hole is between about 7.07 cm² and 7.85*10⁻³ cm² in area. In some embodiments of the kit with the insert, the hole is between about 1.5 cm² and 7.85*10⁻³ cm² in area. In some embodiments of the kit with the insert, the hole is between about 1 cm² and 7.85*10⁻³ cm² in area. In some embodiments of the kit with the insert, the hole is located a distance of more than about 5 mm and less than about 10 cm from a nozzle in the metered dose inhaler when the insert is inserted in place in the opening of the mouth piece of the metered dose inhaler.

In some embodiments, the kit with the cap further comprises a canister that contains a cannabis and/or hemp extract. In some embodiments, the kit with the cap further comprises a metered dose inhaler. In some embodiments, the kit with the insert further comprises a canister that contains a cannabis and/or hemp extract. In some embodiments, the kit with the insert further comprises a metered dose inhaler.

In some embodiments, as noted above, any one of the d and/or l dimensions can also be achieved directly through the molding process of the MDI itself, such as by altering the outer wall 40 of the MDI.

It is noted that, in some embodiments, FIGS. 2 and 3, the outer wall 40, as designated, can depict the outer wall forming the restricted opening 300, and in such embodiments, line 32 does not designate any actual structure (that is, it can be removed from the figure). However, in other embodiments, line 32 can represent the border of the cap 31, and in such embodiments, 40 depicts the outer wall of the MDI that is underneath the cap 31 (as the cap 31 covers it in some such embodiments).

In some embodiments, the MDI can be made of any suitable material. In some embodiments, the MDI can be made from glass, vinyl, glass coated with vinyl, steel, steel coated with vinyl, stainless steel, stainless steel coated with vinyl, aluminum, or aluminum coated with vinyl. In any of the embodiments provided herein, the MDI can be a solution based MDI. In any of the embodiments provided herein, the canister can be a solution based MDI canister.

In some embodiments, any of the restricted openings 300 provided herein can be 1 mm to 1.5 cm in diameter, for example 1 mm to less than 1.5 cm in diameter for example for example 1 mm to 1 cm in diameter. In some embodiments, any of the restricted openings 300 provided herein can have an area of less than 1.5 cm², for example 1.4, 1.3, 1.2, 1.1, 1, 0.9, 0.8, 0.7, 0.6, 0.5, 0.4, 0.3, 0.2, 0.1, 7.85*10⁻³ cm², including any range using any of these values to a value that is lower (e.g., 0.9 or less, 0.8 or less, etc.).

In some embodiments, the additional mass noted in the arrangement in FIG. 1 as 22a, 22b, and 22c can be present with any of the other embodiments provided herein. In other embodiments, items 22a, 22b, and 22c are not present. In some embodiments, the configuration denotes an increased mass of the external geometry of the actuator structure to act as a heat sink. In some embodiments, this is at least 5% additional mass beyond a traditional device. In some embodiments, this is at least 5, 7, 8, 9, 10, 20, 30, 40, 50, 60, 70, 80, 90, 100, 200, 300, 400, 500, 600, 700, 800, 900, or 1000% more mass. Some embodiments of the areas and shapes of the additional mass are shown as item 22a-22c in FIG. 1. In some embodiments, all three areas can employ additional mass. In some embodiments, the area 22a beneath the orifice 21 can employ the additional mass. In some embodiments, the area 22b above the orifice 21 can employ the additional mass. In some embodiments the area 22c behind the orifice 21 can employ the additional mass.

In some embodiments, the checkerboard pattern on FIG. 1 represents options to increase this mass by introducing a cone shape as opposed to a cylinder shape, adding between 0 and 50% to the mass of the valve stem mating boss 75. In some embodiments, the valve stem mating boss is not cylindrical, so that additional mass can be added. In some embodiments, it is cone shaped. In some embodiments, the valve stem mating boss is at least 5, 7, 8, 9, 10, 20, 30, 40, 50, 60, 70, 80, 90, 100, 200, 300, 400, 500, 600, 700, 800, 900, or 1000% more mass than a normal valve stem mating boss (the candy striped section depicted in FIG. 1). In some embodiments, the valve stem mating boss weighs as least 50 mg and less than 5 grams. In some embodiments, the valve stem mating boss 75 has a higher volume than a traditional valve stem mating boss, for example 10, 20, 30, 40, 50, 100, 200, 300, 400, 500, or greater percent increase in volume compared to traditional valve stem mating bosses. In some embodiments, the range of volumes can be 0.15 cm³ to 2.5 cm³. As noted above, in some embodiments, none of the added mass and/or surface area need be present in any of the embodiments provided herein.

In some embodiments, the increased surface area is achieved by increasing the jet length 10 of the cylinder or a shape of the actuator orifice outlet 21.

In some embodiments, the increased surface area is achieved by increasing the texture of the cylinder area by using cuts, grooves, ridges, or by using a mold etched texture such as Mold-Tech MT-11200 of the path through which the formulation passes (from the valve mating boss 75 through to the orifice 21. Thus, in some embodiments, the channel 12 and/or jet 11 can include any additional structural feature so as to increase the surface area of the flow path of the formulation. In some embodiments, the flow path (the path between the valve mating boss and the orifice diameter, is not smooth. In some embodiments, the flow path from the orifice to the exit in the mouth piece is also configured so as to provide an increase in surface area interaction of the formulation with the device. As such, the increased surface area achieved by increasing the texture by using cuts, grooves, ridges, or by using a mold etched texture can also be applied to the interior surface of the outer walls of the MDI 40, in the area of the opening for the mouth piece 30.

In some embodiments, the orifice diameter 20 of the orifice 21 is about 0.15 mm to 0.65 mm, for example 0.2, 0.3, 0.4, 0.5. 0.6, or 0.65 mm, including any range defined between any two of the previous values. In some embodiments, the orifice diameter is greater than 0.3 mm. In some embodiments, the orifice diameter is between 0.3 and 0.7 mm, for example, 0.31, 0.35, 0.4, 0.45, 0.5, 0.55, 0.6, 0.65, or 0.7, including any range defined between any two of the preceding values.

In some embodiments, the actuator jet length has a length of 0.15 to 2.50 mm, for example, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, or 2.5 mm, including any range defined between any two of the previous values.

In some embodiments, a MDI valve is located between the MDI canister and the valve mating boss 75, wherein the MDI valve is configured to dispense between 25 and 125 microliters of formulation per actuation, and wherein the MDI valve is made of at least one of polyoxymethalate (POM), polybutylterephalate (PBT), ABS, acrylic, polycarbonate, ethylene propylene diene monomer (EPDM), or silicon.

In some embodiments, a MDI canister is provided that can include any of the formulations provided herein. In some embodiments, a coating over an interior surface of the canister can also be provided. The coating can be one to increase the level of comfort to a subject to which the formulation is administered. In some embodiments, the coating can alter the taste and/or prevent the taste of the formulation from being altered from storage/exposure to the interior of the canister. In some embodiments, the coating comprises at least one of anodized aluminum or a fluoropolymer. In some embodiments, the fluoropolymer has either been sprayed on the interior surface or deposited via plasma deposition. In some embodiments, the canister further comprises a MDI valve that is configured to dispense between 25 and 125 microliters of formulation per actuation. In some embodiments, the MDI valve comprises at least one of polyoxymethalate (POM), polybutylterephalate (PBT), ABS, acrylic, polycarbonate, ethylene propylene diene monomer (EPDM), and silicon.

By increased “comfort” what is denoted is that the subject feels less discomfort from the inhalation of the formulation, in comparison to when the aspect is absent. In some embodiments, this indicates that the level of comfort is closer to the sensation of normal breathing. In some embodiments, this indicates that the inhaled formulation feels warmer or is less cold, bright or sharp when inhaled.

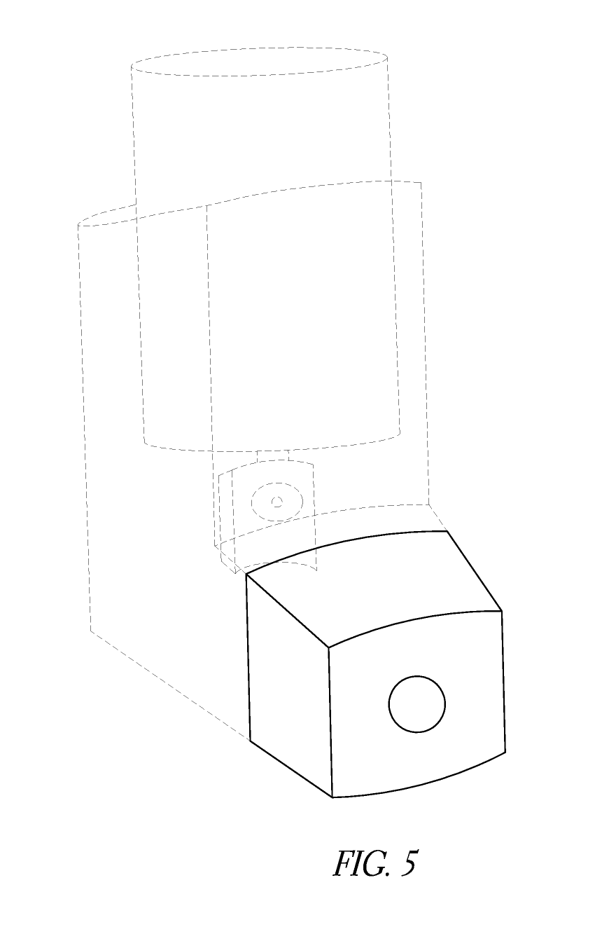

In some embodiments, an ornamental design for a cap for a metered dose inhaler and/or for a metered dose inhaler is provided, as shown in FIG. 5. The broken lines showing a canister in FIG. 5 are included for the purpose of illustrating environment and, in some embodiments, form no part of the claimed design. In some embodiments, an ornamental design for a cap for a metered dose inhaler and/or for a metered dose inhaler is provided, as shown in FIG. 1. In that depiction, line 32 is optional, and need not be present in all embodiments.

In some embodiments, a metered dose inhaler is provided comprising an actuator that comprises a receiver for a canister, an orifice, and a channel fluidly connecting the receiver for the canister to the orifice such that a fluid emitted from the canister is channeled through the channel and out of the orifice to and out of a mouth piece, wherein the mouth piece comprises an opening, wherein the opening is a circular opening between about 1 mm to 3 cm in diameter. In some embodiments, the opening is a circular opening between about 1 mm to 1.5 cm in diameter. In some embodiments, the circular opening is between about 7.07 cm² and 7.85*10⁻³ cm² in area. In some embodiments, the circular opening is between less than 1.5 cm² and 7.85*10⁻³ cm² in area. In some embodiments, the opening is located a distance of more than about 5 mm and less than about 10 cm from the nozzle. In some embodiments, the opening can be square or diamond shaped or oval. In some embodiments, the opening can be any shape and can be 90% or less the area of a standard opening for a MDI, such as less than any of the following: 90, 80, 70, 60, 50, 40, 30, 20, or 10%.

In some embodiments, the metered dose inhaler further comprises a metered dose inhaler canister operably linked to the receiver for the canister. In some embodiments of the metered dose inhaler, the canister further comprises a MDI valve that is configured to dispense between 25 and 125 microliters of formulation per actuation. In some embodiments, the MDI valve is configured to dispense a fixed volume per unit time of formulation per actuation.

In some embodiments of the metered dose inhaler, the MDI valve comprises at least one of polyoxymethalate (POM), polybutylterephalate (PBT), ABS, acrylic, polycarbonate, ethylene propylene diene monomer (EPDM), and silicon.

In some embodiments of the metered dose inhaler, the canister comprises a formulation, said formulation comprising an amount of an extract from a cannabinoid containing plant. In some embodiments, the extract comprises: an activated cannabinoid, wherein the activated cannabinoid comprises at least Tetrahydrocannabinol (THC) and Cannabidiol (CBD); and an amount of a wax, wherein the amount of wax is at least sufficient to provide a higher level of comfort to a subject receiving a dose from a metered dose inhaler that is administering the formulation, and wherein the wax comprises a wax that naturally occurs in a cannabis plant; and an amount of a polar solvent; and an amount of a HFA propellant, wherein the propellant is 1,1,1,2-Tetrafluoroethane (HFA 134a). In some embodiments of the metered dose inhaler, the extract comprises: an activated cannabinoid, wherein the activated cannabinoid comprises at least Tetrahydrocannabinol (THC) and Cannabidiol (CBD), and an amount of a wax, wherein the amount of wax is at least sufficient to provide a higher level of comfort to a subject receiving a dose from a metered dose inhaler that is administering the formulation, and wherein the wax comprises a wax that naturally occurs in a cannabis plant; and an amount of a polar solvent, and an amount of a HFA propellant, wherein the propellant is 1,1,1,2,3,3,3-Heptafluoropropane (HFA 227).

In some embodiments of the metered dose inhaler, the canister comprises a formulation, said formulation comprising an amount of an extract from a cannabinoid containing plant. In some embodiments of the metered dose inhaler, the extract comprises: an activated cannabinoid, wherein the activated cannabinoid comprises at least Tetrahydrocannabinol (THC) and Cannabidiol (CBD), and an amount of a natural terpene, wherein the natural terpene is one that is present in a cannabis plant, and wherein the amount of the natural terpene is at least sufficient to provide a higher level of comfort to a subject receiving a dose from a metered dose inhaler that is administering the formulation, and an amount of a polar solvent, and an amount of a HFA propellant, wherein the propellant is 1,1,1,2-Tetrafluoroethane (HFA 134a). In some embodiments of the metered dose inhaler, the extract comprises: an activated cannabinoid, wherein the activated cannabinoid comprises at least Tetrahydrocannabinol (THC) and Cannabidiol (CBD), and an amount of a natural terpene, wherein the natural terpene is one that is present in a cannabis plant, and wherein the amount of the natural terpene is at least sufficient to provide a higher level of comfort to a subject receiving a dose from a metered dose inhaler that is administering the formulation, and an amount of a polar solvent, and an amount of a HFA propellant, wherein the propellant is 1,1,1,2,3,3,3-Heptafluoropropane (HFA 227).

In some embodiments of the formulation, the ratio of extract:polar solvent: HFA propellent is in a range of 0.5:0.5:99 to 30:30:40. In some embodiments of the formulation, the ratio of extract:polar solvent:HFA propellent is 0.5:0.5:99. In some embodiments of the formulation, the ratio of extract:polar solvent:HFA propellent is 30:30:40. In some embodiments, the formulation further comprises a surfactant. In some embodiments, the formulation further comprises a surfactant at up to 10% w/w.

In some embodiments, a method of restricting a dose supplied by a metered dose inhaler is provided, the method comprising passing a dose supplied by a metered dose inhaler through a restricted opening, wherein the restricted opening has a diameter of less than 3 cm, and wherein a level of discomfort due to the dose is thereby decreased, relative to a level of discomfort that occurs for administration of the dose through an opening that has a diameter of more than 3 cm. In some embodiments, the diameter is less than 1 cm. In some embodiments, the diameter is less than 0.5 cm. In some embodiments, the diameter is less than 0.3 cm. In some embodiments, the diameter is about 0.1 cm.

In some embodiments, a method of restricting a dose supplied by a metered dose inhaler is provided. The method comprises passing a dose supplied by a metered dose inhaler through a restricted opening, wherein the restricted opening has an area of less than 7.07 cm², and wherein a level of discomfort due to the dose is thereby decreased relative to a level of discomfort that occurs for administration of the dose through an opening that has an area of more than 7.07 cm². In some embodiments, a method of restricting a dose supplied by a metered dose inhaler is provided. The method comprises passing a dose supplied by a metered dose inhaler through a restricted opening, wherein the restricted opening has an area of less than 1.5 cm², and wherein a level of discomfort due to the dose is thereby decreased relative to a level of discomfort that occurs for administration of the dose through an opening that has an area of more than 1.5 cm².

In some embodiments, the diameter is less than 0.79 cm². In some embodiments, the diameter is less than 0.2 cm². In some embodiments, the diameter is less than 0.071 cm². In some embodiments, the diameter is about 7.85*10⁻³ cm².

In some embodiments, a method for decreasing discomfort from an application of a dose from a metered dose inhaler is provided. The method comprises providing an altered opening over a mouth piece for a metered dose inhaler, wherein the altered opening is between about 1 mm to 3 cm in diameter. The method comprises providing an altered opening over a mouth piece for a metered dose inhaler, wherein the altered opening is between about 1 mm to less than 1.5 cm in diameter. In some embodiments, the altered opening is between about 7.07 cm² and 7.85*10⁻³ cm² in area. In some embodiments, the altered opening is between about 1 cm² and 7.85*10⁻³ cm² in area. In some embodiments, the altered opening is located at distance of more than about 5 mm and less than about 10 cm from a nozzle within the metered dose inhaler.

### Food Applicator Embodiments

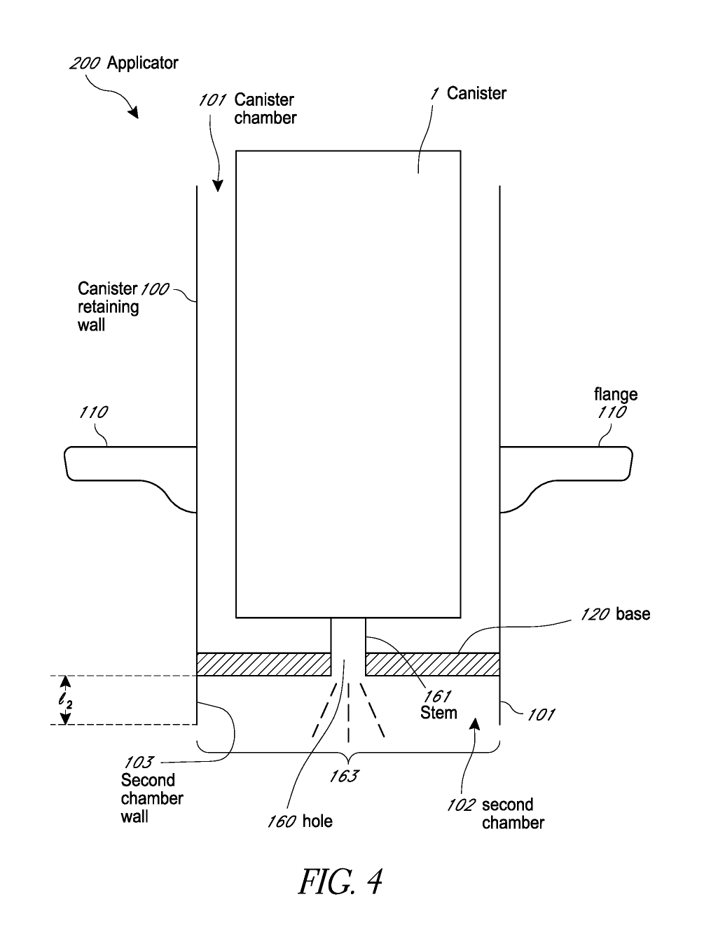

In some embodiments, an applicator 200 is provided, as shown in FIG. 4. In some embodiments, this allows for canister 1 that contains a propellant to be used as a source of material to be applied to other substances, such as food. In some embodiments, the applicator 200 can have a canister retaining wall 100, which can be a structure configured to retain a canister 1 at a desired location. In some embodiments, the applicator 200 can include flanges 110, to allow a user to hold and manipulate the applicator 200, and grip the applicator 200 while depressing the canister to release the propellant driven material within the canister. In some embodiments, the canister can be positioned within the applicator in a canister chamber 101, which can be formed by the canister retaining wall 101 and a base 120. The base 120 can include a hole 160, though which a stem 161 of the canister can fit, so as to allow a formulation within the canister 1 to exit the canister and leave the applicator 200 via a second chamber 102. The second chamber 102 is open to the outer environment and allows the formulation to exit the device in a relatively controlled and/or directed manner and assists in directing the formulation to an area desired by the user. In some embodiments, there can be an additional step within or surrounding the hole 160 for the stem to press against and/or an inner ring. In some embodiments, the hole can have a diameter between 0.1 mm and 3 mm.

The second chamber 102 can be formed by a second chamber wall 103, which can have a length l₂. The length l₂ can vary depending upon the particular desired use. In some embodiments, the length l₂ is from 1 mm to 10 cm, for example 0.1, 0.2, 0.1, 0.4, 0.5, 0.6, 0.7, 0.8, 0.9, 1, 2, 3, 4, 5, 6, 7, 8, 9 or 10 cm.

The various walls and chambers can be made from any suitable material, for example, glass, vinyl, glass coated with vinyl, steel, steel coated with vinyl, stainless steel, stainless steel coated with vinyl, aluminum, or aluminum coated with vinyl.

The various walls and chambers can be continuous walls (for example, without holes or gaps), or can include various holes or gaps in and/or through the surface of the walls.

In some embodiments, the walls are replaced with retaining straps or bands. Thus, in some embodiments, the applicator need only be configured to retain a canister and allow one to deploy the formulation within the canister where desired. In some embodiments, the canister retaining walls 100 can be replaced by one or more rods that connect to the base 120. In some embodiments, the canister retaining wall 100 is tall enough to as to allow the base 120 to be kept functionally associated with the canister (so that the base 120 can apply pressure to the stem 161, so as to allow the release of the formulation from the canister). In some embodiments, the applicator 200 need only include a base 120 with a hole 160 that is sized so as to fit over the stem 161 snuggly enough to allow for the opening of the canister 1 by compressing the base 120 towards the back of the canister 1.

In some embodiments, the shape of the second chamber wall 103 can be varied depending upon its desired use. In some embodiments, the second chamber wall 103 can form a nozzle, so as to direct and/or control the flow of the material emitted from the canister. In some embodiments, the second chamber wall 103 is tapered down as one moves distally from the canister, so that a formulation from the canister is directed more directly beneath the apparatus, and less goes out to the sides of the exit 163 of the second chamber 102.

In some embodiments, one or more flanges 110 can be present in the applicator 200. The flanges can be configured to allow ease of grip and manipulation of the device. The flange 110 can also allow additional pressure to be applied between the back of the canister and the base 120, to allow for ease of opening/release of the material in the canister. In some embodiments, an outer surface of the canister retaining wall 100 is rough, elastic, or ridged so as to allow a better grip on the device.

In some embodiments, any of the formulations provided herein can be contained within the canister 1. In some embodiments, the canister comprises a HFA propellant and ethanol formulation of hemp and/or cannabis. In some embodiments, the formulation is configured for use as a food supplement.

In some embodiments, a method of applying a food supplement is provided. Using one of the applicators provided herein, one can insert a canister containing the formulation to be applied to food and apply pressure between the back of the canister 1 and the base 120 by applying a compressive force between the back of the canister and the flanges 110. Each application will apply a generally consistent dose, so as to allow one to accurately meter out how much extract is desire by repeated compressions. This also can allow one to deposit only oil on the food in order to ingest an accurate amount of concentrate.

In some embodiments, the material can be applied to food, including, without limitation, raw fruits, raw vegetables, cooked vegetables, cooked fruits, processed food, processed food, processed food prior to being canned, potable liquids, meat, meat products, and dairy products.

In some embodiments, an applicator is provided. The applicator comprises: a canister retaining wall configured to receive and retain a canister, wherein the canister is a metered dose inhalation canister, and a base comprising a hole. The hole is configured to receive a stem of the metered dose inhalation canister in a manner such a force can be applied between the base and a back of the canister, which will result in an actuation of the metered dose inhalation canister, and to allow for a dose from the metered dose inhalation canister to pass through the hole. The base is operably coupled to the canister retaining wall such that a force applied to the canister retaining wall is transferred to the base. In some embodiments, this allows for ease of actuation of the stem portion of the canister.

In some embodiments, the applicator further comprises a second chamber, wherein the second chamber is formed by the base and a second chamber wall that at least partially surrounds a perimeter of the base, and wherein the second chamber is open so as to allow the dose to pass to an outside environment.

In some embodiments, the applicator further comprises at least one flange that is attached to the canister retaining wall, wherein a compressive force applied between the at least one flange and a back of the metered dose inhalation canister results in the actuation of the metered dose inhalation canister.

In some embodiments, the applicator further comprises a canister, wherein the canister comprises a hemp extract.

In some embodiments, a method of applying a hemp and/or cannabis extract onto a food product is provided, the method comprising: providing a metered dose inhalation canister containing a hemp and/or cannabis extract; and actuating the canister so that a dose that is released from the canister upon actuation is distributed to a food product. In some embodiments of the method, the metered dose inhalation canister is positioned within an applicator. In some embodiments of the method, the applicator comprises a base with a hole that is sized to receive a stem of the metered dose inhalation canister. In some embodiments of the method, the applicator further comprises a canister chamber that is configured to retain the metered dose inhalation canister. In some embodiments of the method, the applicator further comprises a flange that is attached to an outside surface of the canister chamber. In some embodiments of the method, actuating the canister is achieved by squeezing the flange towards an end of the metered dose inhalation canister that is opposite of the stem. In some embodiments, the actuation is repeated as many times as desired. In some embodiments, the method can be applied with any of the applicators provided herein.

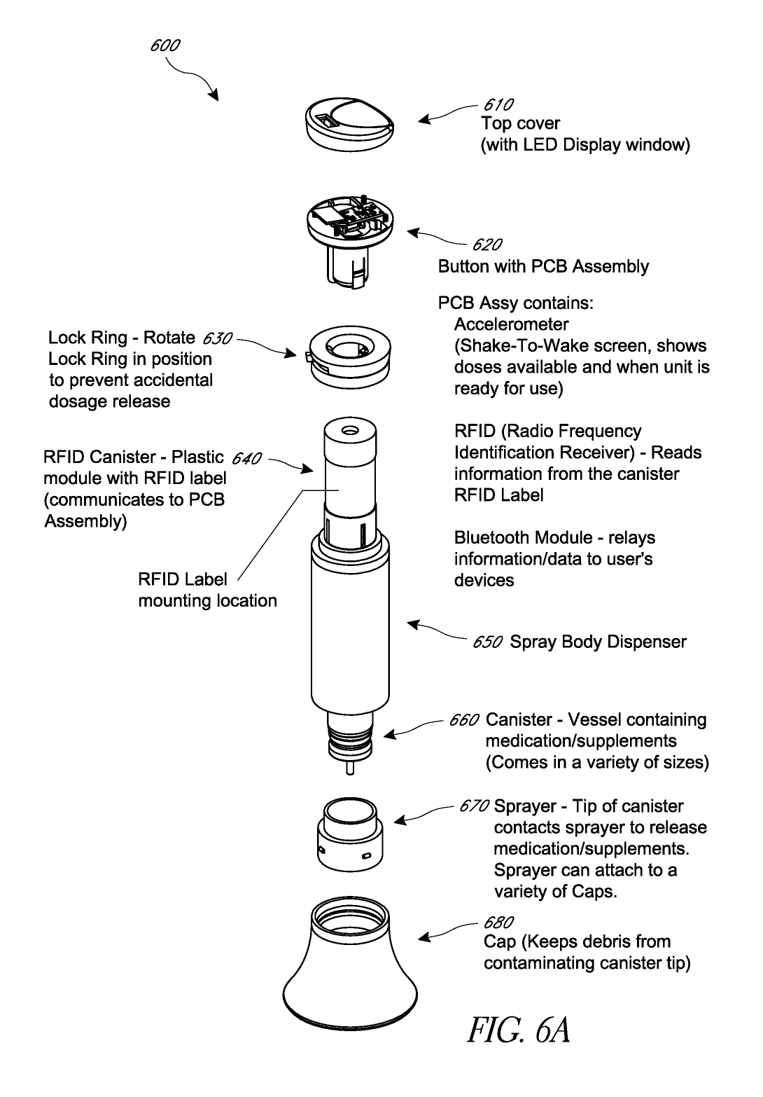

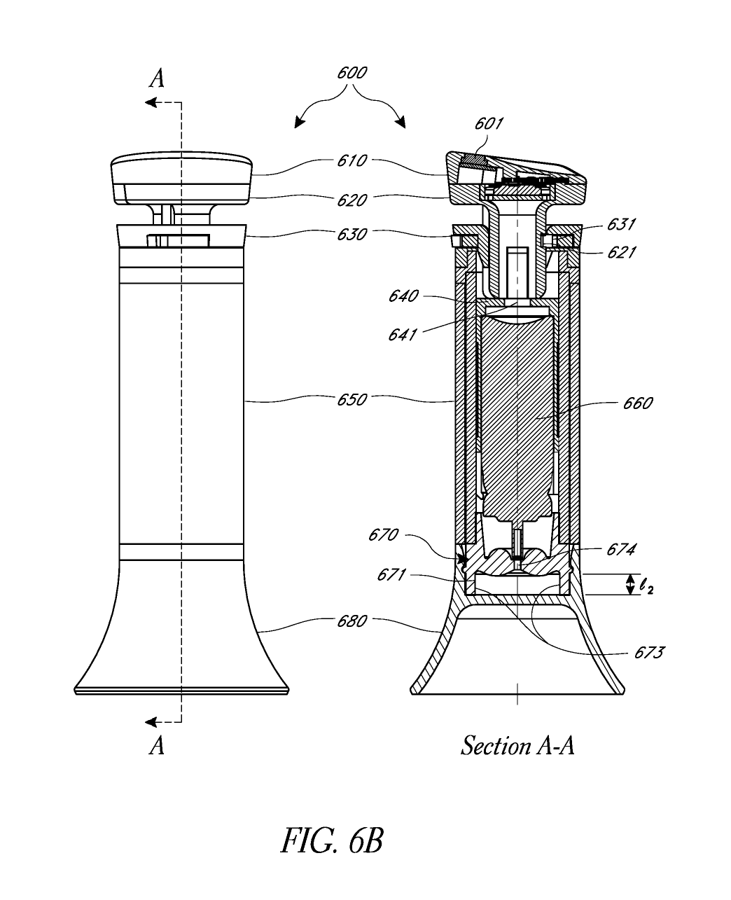

FIGS. 6A and 6B display additional embodiments of food applicators. FIG. 6A displays a food applicator 600 in an exploded arrangement and FIG. 6B displays the parts assembled and with a sectional view A-A. In some embodiments, the food applicator 600 can include a top cover 610 with an optional display 601 (which can be a LED display). The top cover 610 can be configured to allow for the ease of actuation and placement of a finger or thumb. Beneath the top cover 610 is the button mechanism 620, which can include a PCB (printed circuit board) assembly. The PCB assembly can include an accelerometer (shake-to-wake screen). It can also be configured to display, via the LED display 601, the doses available, and when the unit is ready for use. The PCB assembly can also include a RFID (radio frequency identification receiver), which can be configured to read information from a RFID chip (641 FIG. 6B). The PCB assembly can also include a Bluetooth module, which can relay information/data to another device. In some embodiments, the electronics in the PCB assembly can include a memory storage system and/or a microprocessor. In some embodiments, the PCB assembly can be positioned in something other than the button, for example, the top cover 610 or the pray body dispenser 650.

The food applicator 600 can further include a lock ring 630, which allows one to prevent accidental dosage release. In some embodiments, the button 620 has an indentation 621, into which a locking member 631 can be inserted, so as to prevent actuation of the button. The food applicator 600 can further include a RFID canister 640. The RFID canister 640 can be a plastic module with a RFID label, which can be in communication with the PCB assembly 620. In some embodiments, any locking system can be employed to reduce the risk of inadvertent actuation. The RFID canister 640 can include a RFID chip 641, which can be positioned anywhere within the RFID canister 640. In some embodiments, the RFID chip 641 can be positioned at the back of the RFID canister 640. In some embodiments, there are electrical leads or contacts on the button and/or the RFID chip 641, so that actuations can be counted and displayed. In some embodiments, the RFID canister 640 is a plastic sleeve that slides over the canister 660 that contains the formulation. In some embodiments, the RFID chip is part of or attached directly to the canister 660 itself.

The food applicator 600 can also include a spray body dispenser 650, into which the canister 660 containing the formulation can be inserted and retained. In some embodiments, the food applicator includes a sprayer 670, which the tip of the canister 660 contacts when in use to release the formulation. The sprayer 670, is configured to be attached to a cap 680, which assists in keeping debris and contaminants from touching the canister tip. In some embodiments, the sprayer 670 can be the same configuration or arrangement and provide the same function as the base 120 in FIG. 4.

In some embodiments, the cap 680 is configured to allow the food applicator to stand upright. In some embodiments, the cap 680 is configured to seal the sprayer 670 from and contaminants. In some embodiments, the cap 680 leaves the sprayer 670 open to the environment, but keeps the sprayer 670 distanced from other objects. In some embodiments, the sprayer 670, has the same arrangements provided herein regarding the underside of the embodiment in FIG. 4. For example, the sprayer 670 can include a length “l” of a wall 671, a hole 674, and an exit 673.

In some embodiments, the food applicator is used to dispense one or more of the formulations provided herein. In some embodiments, the food applicator is used to dispense medications, nutrients, and/or supplements. In some embodiments, any material can be dispensed, if it can be effectively put into a canister.

In some embodiments, the cap 680 is configured to be removably attached to the sprayer 670. In some embodiments, the sprayer 670 is configured to be removably attached to the spray body dispenser 650. In some embodiments, the spray body dispenser 650 is configured and sized to receive a canister and to keep the canister securely positioned therein. In some embodiments, the lock ring 630, is removably attached to the spray body dispenser 650. Any system of removable attachment can be employed, as long as it allows for the use of the food applicator. In some embodiments, one can use pressure fittings, threaded arrangements, snapping arrangements (such that one side is grooved and the other side has a protrusion that fits into said groove etc. as removable attachment. In some embodiments, the RFID canister 640 is configured and sized to hold and retain the canister 660. In some embodiments, the inside of the spray body dispenser 650 is configured and sized to hold and removably retain the RFID canister 640, while it retains the canister 660. In some embodiments, placement of the canister 660 into the RFID canister 640, effectively locks the canister 660 in the RFID canister 640.

Formulations for Use with the MDI and Food Applicators

In some embodiments, any formulation comprising hemp and/or cannabis extract can be used in either and/or both of the MDI embodiments and the food applicator embodiments. In some embodiments, food grade oil can be in the canisters to be used in the applicators, in addition to the hemp and/or cannabis. In some embodiments, other food flavorings can be added to the canister to be used in the applicator. In some embodiments, HFA is used to deposit a clean layer of nutritional oil atop food without any residue from carries suck as (glycol, alcohol, water, Polyethylene glycol, of glycerin). In some embodiments, any of the formulations provided herein regarding hemp and/or cannabis can have the hemp and/or cannabis replaced (either in part or in whole) with any nutritional material, any supplement material, and/or any other natural product as long as it can be distributed from a canister. In some embodiments, the formulation in the canister for the food applicator can comprise a biosynthetic cannabinoid, for example a cannabinoid from yeast or artificial sources. In some embodiments, the formulation to be used for the food applicator can comprise or consist of synthetic and/or biosynthetic (from engineered yeasts and such) sources of >95% (w/w) purity of cannabinoids that are naturally found in cannabis (THC, CBD, THCV, CBV, etc).[0076] In some embodiments, the formulation can comprise an amount of at least one cannabinoid, an amount of a propellant suitable for metered dose inhalation application to a human subject, a polar solvent miscible with the propellant, a cannabinoid(s) and/or hemp, and an amount of a wax and/or a terpene.

In some embodiments, the wax can be any type of wax. In some embodiments, the wax is a natural wax. The wax can comprise a plant based natural wax. The plant based natural wax can comprise a cannabis wax. In some embodiments, the plant based natural wax can comprise a non-cannabis wax. In some embodiments, the cannabis wax can include one or more of a straight chain paraffins such as nonacosane (C29), heptacosane (C27) or hentriacontane (C31), or branched chained parafins such as isononacosane, isoheptacosane or isohentriacontane.

In some embodiments, the formulation can further (or instead) comprise at least one terpene. In some embodiments, any type of terpene can be employed. In some embodiments, the amount of the terpene is at least 3 mcg/mL and less than 100 mg/mL. In some embodiments, the terpene is selected from the group of at least one of: Pinene, Limonene, Myrcene, Phellandrene, Carene, Terpinene, Linalool, Fenchol, Borneol, Terpineol, Geraniol, Humulene, Caryophyllene, Bisabolol or Phytol.

In some embodiments, the amount of the at least one cannabinoid is sufficient for the desired purpose (for example achieving great subject comfort). In some embodiments, the amount of the at least one cannabinoid is at least 0.2 mg/mL and less than 350 mg/mL, for example 0.5 to 300 mg/mL, 1 to 200 mg/mL, 10 to 100, or 30 to 60 mg/mL.

In some embodiments, the cannabinoid employed can be 100% activated. In some embodiments, less than 100% of the cannabinoid present is activated, for example, less than 100%, 99, 98, 95, 90, 80, 70, 50, 40, 30, 20, 10, or 5% or less of the cannabinoid present can be activated. In some embodiments, at least 50% of the cannabinoid is activated, for example, at least 50, 60, 70, 80, 90, 95, 96, 97, 98, or 99 percent of the cannabinoid is activated. In some embodiments, none of the cannabinoid is activated. In some embodiments the at least one cannabinoid comprises at least one activated cannabinoid, such as 2, or more different cannabinoids are activated and/or at least partially activated.

The term “cannabinoid” as used herein denotes a class of diverse chemical compounds that act on cannabinoid receptors on cells that repress neurotransmitter release in the brain. The class includes endocannabinoids, phytocannabinoids, and synthetic cannabinoids. Examples include cannabis-derived cannabindoids, delta-nine-tetrahydrocannabinol (THC) and cannabidiol (CBD), Cannabigerol-type, Cannabichromene-type, Tetrahydrocannabinol- and Cannabinol-type, Cannabielsoin-type, iso-Tetrahydrocannabinol-type, Cannabicyclol-type, and Cannabicitran-type. In some embodiments, cannabinoid includes: CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), and/or CBGM (Cannabigerol Monomethyl Ether).

In some embodiments, the at least one cannabinoid can be at least one of tetrahydrocannabinol (THC), cannabidiol (CBD), and tetrahydrocannabivarin (THCV). In some embodiments, the at least one cannabinoid comprises both THC and CBD.

In some embodiments, the formulation further comprises at least one terpene. In some embodiments, in addition to the at least one terpene noted above, the formulation comprises at least one activated cannabinoid. In some embodiments, the at least one terpene comprises at least caryophyllene. In some embodiments, the at least one terpene is present in an amount of at least about 3 mcg/mL, for example, at least 4, 5, 6, 7, 8, 9, 10, 20, 30, 40, 50 or more mcg/mL.

In some embodiments, the amount of wax present in the formulation is greater than 10 micrograms/mL and less than 50 mg/mL. In some embodiments, the wax is present in at least 0.03 mg/mL in the final formulation.

In some embodiments, the natural carbon dioxide cannabis extract has <10 ppm of residual solvent. In some embodiments, the solvent comprises an amount of ethanol, methanol, butane, and/or isopropanol. In some embodiments, the ethanol is present in an amount of up to 30% w/w, for example, up to 5, 10, 15, 20, 25, or 30% w/w.

A variety of propellants can be employed in various embodiments. In some embodiments, the propellant comprises at least a HFA. In some embodiments, the HFA is at least one of 1,1,1,2-Tetrafluoroethane (HFA 134a) and 1,1,1,2,3,3,3-Heptafluoropropane (HFA 227). In other embodiments, alternative solvents can be employed.

In some embodiments, the formulation further comprises a surfactant. In some embodiments, the surfactant is present in an amount adequate for improving solubility. In some embodiments, the surfactant is present in an amount of up to 10% w/w. In some embodiments, the surfactant comprises oleic acid. In some embodiments, additional or alternative surfactants can be applied if confirmed for the uses as provided herein.

In some embodiments, the formulation can comprise an amount of an activated cannabinoid, wherein the activated cannabinoid comprises at least Tetrahydrocannabinol (THC) and Cannabidiol (CBD), an amount of ethanol sufficient to serve as a solvent, and an amount of a propellant, wherein the propellant is at least one of 1,1,1,2-Tetrafluoroethane (HFA 134a) and 1,1,1,2,3,3,3-Heptafluoropropane (HFA 227). In some embodiments, the formulation can also include at least one of: a) an amount of a wax, wherein the amount of wax is at least sufficient to provide a higher level of comfort to a subject receiving a dose from a metered dose inhaler that is administering the formulation, and wherein the wax comprises a wax that naturally occurs in a cannabis plant, or b) an amount of a natural terpene. The natural terpene is one that is present in a cannabis plant, and wherein the amount of the natural terpene is at least sufficient to provide a higher level of comfort to a subject receiving a dose from a metered dose inhaler that is administering the formulation. In some embodiments, the wax is one that is natural to a cannabis plant, but is not removed fully during the processing of the cannabis plant.

In some embodiments, a formulation for metered dose inhalation is provided. In some embodiments, the formulation can comprise 1) an amount of an extract from a cannabinoid containing plant. The extract can comprise an activated cannabinoid, wherein the activated cannabinoid comprises at least Tetrahydrocannabinol (THC) and Cannabidiol (CBD). The formulation can further include at least one of: i) an amount of a wax, wherein the amount of wax is at least sufficient to provide a higher level of comfort to a subject receiving a dose from a metered dose inhaler that is administering the formulation, and wherein the wax comprises a wax that naturally occurs in a cannabis plant, or ii) an amount of a natural terpene. The natural terpene can be one that is present in a cannabis plant. The amount of the natural terpene and/or the natural wax is at least sufficient to provide a higher level of comfort to a subject receiving a dose from a metered dose inhaler that is administering the formulation. The formulation can further include 2) an amount of a polar solvent; and 3) an amount of a HFA propellant, wherein the propellant is at least one of 1,1,1,2-Tetrafluoroethane (HFA 134a) and 1,1,1,2,3,3,3-Heptafluoropropane (HFA 227). In some embodiments, the extract:polar solvent:HFA propellent is in a ratio ranging from about 0.5:0.5:99 to about 30:30:40. In some embodiments, the formulation further includes a surfactant at up to 10% w/w.

In some embodiments, the extract containing the cannabinoid can be decarboxylated by immersion in an oil bath. In some embodiments, this occurs at above 100 degrees centigrade for an adequate amount of time. In some embodiments, this occurs for 155 degrees centigrade for 80 minutes.

In some embodiments, the extract can then be mixed an organic solvent. In some embodiments, this can be alcohol. In some embodiments, it is 200 Proof USP grade ethanol. In some embodiments, this can be at a ratio of 37:63 extract to ethanol. Dissolution of the extract can be achieved by agitation and heating. In some embodiments, this can be heated to as much as 78 degrees Centigrade (boiling point of ethanol) to fully dissolve the extract. The sample is then cooled. In some embodiments, it is cooled to 5 degrees centigrade to form a gel phase. In some embodiments, this can be employed in a suitable MDI canister. The canister is crimped with a suitable valve and charged with 134a (or 227ea) by over pressure through the valve stem. The canister is agitated by hand for no less than 10 seconds.

In some embodiments, a method for preparing a formulation can include providing a source of a cannabinoid. The method can further include obtaining an active ingredient from the source by carbon dioxide extraction. The method can further include combining the active ingredient with a polar solvent to obtain a mixture. The method can further include heating the mixture to solubilize it into a solvent. The method can further include cooling the solvent to thereby obtain a gel or viscous liquid and solubilizing the gel or viscous liquid with HFA.

In some embodiments, the formulation is prepared by preparing the extract in separate fractions. In some embodiments, heating comprises extracting at least one of THC or CBD via an oil bath.

In some embodiments, a metered dose inhaler canister is provided. The canister can include a solution. The solution can include a solvent. In some embodiments, any solvent provided herein can be employed. In some embodiments, the solvent comprises at least one of butane and/or propane. The canister can further include a naturally occurring and/or derived oil (such as an essential oil).

In some embodiments, a metered dose inhaler canister is provided that can include a solvent. In some embodiments, any solvent provided herein can be employed. In some embodiments, the solvent comprises at least one of butane and/or propane. The canister can further include a naturally occurring and/or derived oil (such as an essential oil).

In some embodiments, a metered dose inhaler canister comprising a solution is provided. The solution can comprise at least one of a) a solvent, wherein the solvent comprises at least one of butane or propane and an extract from a cannabinoid containing plant, or b) a solvent having a vapor pressure of 1.5 atmospheres or more at room temperature and an extract from a cannabinoid containing plant.

In some embodiments, the canister can include any of the other components, solutions, ingredients, etc. provided herein.

In some embodiments, an applicator is provided. The applicator comprises a canister retaining wall configured to receive and retain a canister, wherein the canister is a metered dose inhalation canister. The applicator can further include a metered dose inhaler canister and a base comprising a hole. The hole is configured a) to receive a stem of the metered dose inhalation canister in a manner such a force can be applied between the base and a back of the canister, which will result in an actuation of the metered dose inhalation canister, and b) to allow for a dose from the metered dose inhalation canister to pass through the hole. In some embodiments, the base is operably coupled to the canister retaining wall such that a force applied to the canister retaining wall is transferred to the base. Some embodiments of this aspect are depicted in FIG. 4. In some embodiments, the canister can contain any of the solutions provided herein in regard to canisters for metered dose inhalers. In some embodiments, the metered dose inhaler canister contains a solution comprising: a) a solvent, wherein the solvent comprises: at least one of butane or propane; and a naturally occurring and/or derived essential oil, b) a solvent, wherein the solvent comprises: at least one of butane or propane; and an extract from a cannabinoid containing plant, c) a solvent having a vapor pressure of 1.5 atmospheres or more at room temperature (for example 20 degrees Celsius), or d) a solvent having a vapor pressure of 1.5 atmospheres or more at room temperature and an extract from a cannabinoid containing plant.

In some embodiments, the formulation of the food applicator can compromise HFA 134a, USP Grade ethanol, and >99% (w/w) purity CBD with less than 0.3% THC (w/w) and less than 0.1% wax (w/w). For some applications, this can unexpectedly provide a consistent and accurate way to meter a small amount (0.1-5 mg) of medicine per actuation as a thin film onto a substrate to be ingested. Further, this allows patients to accurately titrate cannabinoid consumption. In some embodiments, there is less than 25% w/w and more than 0.5% ethanol in the propellant for the food applicator formulation. In some embodiments, the formulation of the food applicator can compromise HFA 134a, USP Grade ethanol, and >99% (w/w) purity CBD with less than 0.5% THC (w/w) and less than 0.3% wax (w/w). In some embodiments, the formulation of the food applicator can compromise HFA 134a, USP Grade ethanol, and >99% (w/w) purity CBD with less than 0.1% THC (w/w) and less than 0.05% wax (w/w).

In some embodiments, the active being delivered is a whole plant cannabis extract that can be dominant in other cannabinoids such as THC, CBD or THCV.

In some embodiments, the applicator in FIG. 4 can be configured for application of the material in the canister onto a substance for smoking or a “smoking substance”. For example, the size of the hole 160, second chamber wall 103, second chamber 102, and exit 163, can be configured so as to allow application of the material in the canister onto a surface area that is typical of a material to be smoked. This surface area can be of any size, but in some embodiments, it is between the size of an unrolled cigarette (so that, for example, the cigarette paper and/or tobacco on the paper can be sprayed easily) and as small as, for example, the end of a cigarette (so that the oil can be directed to the end of a cigarette. In some embodiments, the applicator can be a metered dose inhaler as described herein.

In some embodiments, the material in the canister includes any desired material. In some embodiments, the desired material can be an oil that provides some desired aspect when smoked or applied to a material to be smoked. In some embodiments, the oil (or a part thereof) is from a naturally derived source and can be, for example, an essential oil. In some embodiments, the oil includes THC, and/or a cannabinoid, and/or cannabidiol, and/or a terpene, and/or a wax. In some embodiments, any propellant can be employed within the canister for the applicator. In some embodiments, any oil can be employed with the propellant in the canister, in the applicator.

In some embodiments, the amount of the ingredient and/or oil is determined such that a set number of actuations of the actuator delivers a desired amount of the ingredient and/or oil onto the material to be smoked. In some embodiments, it is one actuation, in others, it is more, for example, 2, 3, 4, 5, 6, 7, 8, 9, 10 or more actuations. In some embodiments, the amount delivered for each actuation is similar. In some embodiments, the applicator includes a metering valve.

In some embodiments, a method for applying an oil to a smoking substance is provided. The method can comprise employing at least one of a) any of the applicators described herein or b) any of the metered dose inhalers described herein to apply an oil (or material contained therein) onto a smoking substance.

In some embodiments, the oil comprises a hemp and/or cannabis extract or any other natural extract.

In some embodiments, the smoking substance is a cigarette, joint, clove, or material within a pipe.

In some embodiments, a method of applying a hemp and/or cannabis extract onto a smoking product is provided. The method comprises providing a metered dose inhalation canister containing a hemp and/or cannabis extract and actuating the canister so that a dose that is released from the canister upon actuation is distributed onto a smoking product. In some embodiments, the smoking product comprises at least one of tobacco, cloves, or cannabis.

### EXAMPLE 1

A canister containing a cannabis extract is inserted into the MDI of FIG. 2. A subject to receive the cannabis formulation depresses the canister to actuate it, delivering the cannabis formulation to the subject. The subject experiences less discomfort than if the subject had applied a cannabis formulation according to FIG. 2 (with an unrestricted opening of the mouth piece).

### EXAMPLE 2

A canister containing a hemp extract is inserted into the applicator of FIG. 4. A subject positions the applicator over an item of food so that the bottom opening is over the food. The subject then depresses the canister to actuate it, delivering one unit of hemp oil formulation to the surface of the food. If additional hemp oil is desired, the canister can be actuated 2 to 10 additional times, delivering approximately equal amounts of hemp oil to the surface of the food for each actuation.

### EXAMPLE 3

A canister containing a hemp extract is inserted into the applicator of FIG. 4. A subject positions the applicator over tobacco that has been placed on top of cigarette paper so that the bottom opening is over the tobacco and/or the paper. The subject then depresses the canister to actuate it, delivering one unit of hemp oil formulation to the surface of the tobacco and/or the paper. If additional hemp oil is desired, the canister can be actuated 2 to 10 additional times, delivering approximately equal amounts of hemp oil to the surface of the tobacco and/or the paper for each actuation.

1. A metered dose inhaler comprising:
an actuator that comprises a receiver for a canister;
an orifice; and
a channel fluidly connecting the receiver for the canister to the orifice such that a fluid emitted from the canister is channeled through the channel and out of the orifice to and out of a mouth piece, wherein the mouth piece comprises an opening, wherein the opening is at least one of:
a) between about 1 mm to less than 1.5 cm in diameter,
b) between about 1 cm² and 7.85*10⁻³ cm² in area, or
c) located a distance of more than about 5 mm and less than about 10 cm from the nozzle.

2. The metered dose inhaler of claim 1, further comprising a metered dose inhaler canister operably linked to the receiver for the canister.

3. The metered dose inhaler of claim 2, wherein the canister further comprises a MDI valve that is configured to dispense between 25 and 125 microliters of formulation per actuation.

4. The metered dose inhaler of claim 3, wherein the MDI valve comprises at least one of polyoxymethalate (POM), polybutylterephalate (PBT), ABS, acrylic, polycarbonate, ethylene propylene diene monomer (EPDM), and silicon.

5. The metered dose inhaler of claim 2, wherein the canister comprises a formulation, said formulation comprising an amount of an extract from a cannabinoid containing plant.

6. The metered dose inhaler of claim 5, wherein the formulation comprises:
a) an extract, wherein the extract comprises:
a) an activated cannabinoid, wherein the activated cannabinoid comprises at least Tetrahydrocannabinol (THC) and Cannabidiol (CBD);
b) optionally at least one of:
a) an amount of a wax, wherein the amount of wax is at least sufficient to provide a higher level of comfort to a subject receiving a dose from a metered close inhaler that is administering the formulation, and wherein the wax comprises a wax that naturally occurs in a cannabis plant, or
b) an amount of a natural terpene, wherein the natural terpene is one that is present in a cannabis plant, and wherein the amount of the natural terpene is at least sufficient to provide a higher level of comfort to a subject receiving a dose from a metered dose inhaler that is administering the formulation;
c) an amount of a polar solvent; and
d) an amount of a HFA propellant, wherein the propellant is at least one of 1,1,1,2-Tetrafluoroethane (HFA 134a) and 1,1,1,2,3,3,3-Heptafluoropropane (HFA 227), wherein extract:polar solvent:HFA propellent is in a ratio ranging from 0.5:0,5:99 to 30:30:40.

7. The formulation of claim 6, further comprising a surfactant at up to 10% w/w.

8. A method of restricting a dose supplied by a metered dose inhaler, the method comprising passing a dose supplied by a metered dose inhaler through a restricted opening, wherein the restricted opening has a diameter of less than 1.5 cm, and wherein a level of discomfort due to the dose is thereby decreased, relative to a level of discomfort that occurs for administration of the dose through an opening that has a diameter of more than 1.5 cm.

9. The method of claim 8, wherein the diameter is less than 1 cm.

10. The method of claim 8, wherein the diameter is less than 0.5 cm.

11. The method of claim 8, wherein the diameter is less than 0.3 cm.

12. The method of claim 8, wherein the diameter is about 0.1 cm.

13-18. (canceled)

19. An applicator comprising:
a canister retaining wall configured to receive and retain a canister, wherein the canister is a metered dose inhalation canister; and
a base comprising a hole, wherein the hole is configured
a) to receive a stem of the metered dose inhalation canister in a manner such a force can be applied between the base and a back of the canister, which will result in an actuation of the metered dose inhalation canister, and
b) to allow for a dose from the metered dose inhalation canister to pass through the hole, and
wherein the base is operably coupled to the canister retaining wall such that a force applied to the canister retaining wall is transferred to the base,

20. The applicator of claim 19, further comprising a second chamber, wherein the second chamber is formed by the base and a second chamber wall that at least partially surrounds a perimeter of the base, and wherein the second chamber is open so as to allow the dose to pass to an outside environment.

21. The applicator of claim 20, further comprising at least one flange that is attached to the canister retaining wall, wherein a compressive force applied between the at least one flange and a back of the metered dose inhalation canister results in the actuation of the metered dose inhalation canister.

22. The applicator of claim 19, further comprising a canister, wherein the canister comprises a hemp extract.

23. A method of applying a hemp and/or cannabis extract onto a food product, the method comprising:
providing a metered dose inhalation canister containing a hemp and/or cannabis extract; and
actuating the canister so that a dose that is released from the canister upon actuation is distributed to a food product.

24. The method of claim 23, wherein the metered dose inhalation canister is positioned within an applicator.

25. The method of claim 24, wherein the applicator comprises a base with a hole that is sized to receive a stem of the metered dose inhalation canister.

26. The method of claim 25, wherein the applicator further comprises a canister chamber that is configured to retain the metered dose inhalation canister.

27-40. (canceled)
